(19) United States
(12) Patent Application Publication
(10) Pub. No.: US 2014/0214078 A1
(43) Pub. Date: Jul. 31, 2014

(54) Magnetic Wound Closure Assembly and Method of Use

(71) Applicant: Moustafa Moustafa, Beverly Hills, CA (US)

(72) Inventor: Moustafa Moustafa, Beverly Hills, CA (US)

(21) Appl. No.: 13/752,259

(22) Filed: Jan. 28, 2013

(51) Int. Cl. A61B 17/08

(52) U.S. Cl. CPC A61B 17/08
USPC 606/215

(57) ABSTRACT

A wound closure assembly using magnets aids in healing by drawing skin together without using stitches. The assembly has a flexible sheet with two magnet coupling ends. Each magnet coupling end is correspondingly shaped to a channel within each of two channeled magnets. The channeled magnets are coupled to the opposite ends of the flexible sheet, and also have an adhesive layer that adheres the magnets to the patients skin. The practitioner, such as a doctor, places the two magnets on opposite sides of a wound, and uses the flexible sheet to guide and hold the position of each magnet on the patient's skin. The practitioner slidably disengages the flexible sheet from the magnets in the lengthwise direction of the magnet channels, thereby leaving only the magnets around the patient's wound. The magnets on each side of the wound attract each other, thereby reapproximating the wound's edges.

### FIELD OF THE INVENTION

The present invention relates generally to an assembly useful for wound healing, and more particularly to an assembly having magnets that draw wound edges in proximity to each other.

### BACKGROUND OF THE INVENTION

Wounds usually occur when there is trauma to the skin and underlying tissue. Types of trauma include lacerations, abrasions, incisions, punctures, and penetrations. After trauma, the wound begins to heal in a complex series of biochemical processes occurring is several wound healing phases. The phases of healing are often categories into a hemostasis phase, a inflammatory phase, a proliferative phase and the remodeling phase. In hemostasis, active bleeding is controlled by clotting. In the inflammatory phase, pathogens are removed by the body away from the wounded area, and biological factors are released (which later cause the division of cells involved in the proliferative phase). In the proliferative phase, new blood vessels are formed and wound contraction occurs. Also in the proliferative phase, epithelial cells cover the wound, providing an area of growth for new tissue. During contraction, the wound is made smaller by myofibroblasts attaching to the wound edges, and finally, during the remodeling phase, collagen fibers are realigned along tension lines formed during the earlier phases of healing.

Not only is the process of wound healing complex, but it is also fragile, since many factors can lead to a disruption of proper wound healing, including re-injury of the tissue, bacterial infection, and physical stress on the damaged tissue. A variety of devices and methods have been used to aid in the wound healing process. These devices and methods are generally divided into one of three types: primary intention, secondary intention, and tertiary intention. The primary intention devices and methods bring the edges of the wounds together, so that the edges are reapproximated. Reapproximation helps to minimize scarring, and increases the speed at which wound contraction and healing occur. Examples of primary intention devices and methods include the use sutures, staples, tape, glue, and hooks. Primary intention techniques to heal wounds are the most common techniques used by practitioners. While not as commonly used, secondary intention devices and methods include allowing the wound to first granulate without closing the wound and thereafter packed and drained on several occasions to remove debris. Still less common are tertiary intension devices and methods, which delay the closure of the wound even longer, so that the practitioner can close the wound a later time. Tissue grafting is an example of a tertiary method for wound healing.

One problem with not immediately treating a wound is that if the wound edges are not reapproximated soon after injury, healing is delayed. This delay leads to scarring, and infection. While in some circumstances a delay is advantageous, practitioners usually want to close an open wound as soon as possible. Quick and easy devices and methods are therefore needed to reapproximate the wound edges.

The traditional method to reapproximate wound edges is by sutures, where a practitioner stitches a threading material to connect opposing sides of a wound. Sutures and suturing techniques are well known in the prior art, such as described in U.S. Pat. No. 8,267,959. Other devices and methods to reapproximate wound edges include hooking devices, such as the hook closure device in in U.S. patent application Ser. No. 13/266,825 where a band placed over a wound has a multiplicity of hook elements that engage a mesh on the opposing side of a wound.

The use adhesive strips is another method to aid in wound closure. In U.S. Pat. No. 4,825,866, adhesive strips are placed on opposite sides of a wound and drawn together to reapproximate the wound edges. Stapling and clipping the edges of wound are other techniques to reapproximate wound edges, as described in U.S. Pat. No. 7,556,632.

The use of magnets to reapproximate wound edges has also previously been described. U.S. patent application Ser. No. 10/512,964 and 12/721,651 are two applications that have described tissue joining devices comprising interconnecting components where the magnetic components are attracted to each other and draw tissue together using magnetism.

Medical adhesives, such as cyanoacrylate glues provide for very tight, high-strength closure of wounds without the need for the physical closure accomplished with sutures, however, cyanoacrylate based glues have been associated with the formation of toxins byproducts, and even non-toxic versions are really only useful for smaller, shallow lacerations in low-tension areas. These adhesives can be very unforgiving if the practitioner needs to remove the glue. Another disadvantage is that glues can leak into areas such as the eye, where there are serious ramifications, especially if the adhesives are toxic. Still another disadvantage is that adhesives can trap pathogens and other particles within the wound.

Each type of wound closure device and technique has advantages and disadvantages. Sutures pose the risk of needle stick injury to the patient, as well as to health care professions. The process of suturing also can take a substantial amount of time depending on the size of the wound. Using staples for wound closure is more rapid than suturing, however, unlike sutures, which may be absorbed by the body, staples usually have to be removed by a special tool. Sutures and staples also require applying local anesthesia which could be painful and toxic to the patient. Furthermore, if the practitioner needs to enter the wound area, the sutures or staples need to be cut or removed.

Some of the more complex wound closure devices that reduce some of these disadvantages have many individual parts, are difficult to apply, and are expensive. Accordingly, it would be advantageous to make available a novel wound closure assembly that reduces these stated disadvantages.

### SUMMARY OF THE INVENTION

The present invention relates to a wound closure assembly. It is an object of the present invention to provide an assembly of a flexible sheet with two magnets, one magnet on two of the opposing ends of the flexible sheet. By placing two skin adhering magnets on opposing sides of a patient's wound, the magnets attract each other, thereby drawing in the skin around the wound, making the wound opening smaller. The flexible sheet between the magnets helps guide the practitioner to correctly place the magnets on the patient's skin. Once correctly placed, the practitioner disengages the magnets from the sheet by sliding the sheet in an elongate direction along channels within the magnets, thereby leaving only the magnets around the patent's wound. The practitioner can place multiple wound closure

The flexible sheet has a first magnet coupling or connecting end, an opposing second magnet coupling or connecting end, and a central portion between the first and second ends. Attached to the first end of the sheet is a first magnet having an adhesive layer on the magnet skin contact surface. The second magnet also has an adhesive layer on the magnet skin contact surface. Each of the two magnets has an elongated channel that runs the length of the each magnet. The shape of the channel within the magnet is correspondingly shaped to the shape of each coupling end of the flexible sheet. This allows each end of the flexible sheet to be inserted into the channel of each of the magnets, thereby attaching the magnets to flexible sheet.

In one embodiment, the magnets further comprise a removable adhesive cover, which covers the adhesive layer on the contact surface of the magnet. The removable adhesive cover may be a peelable strip that protects the adhesive layer on the magnet. The adhesive cover protects the adhesive layer on the magnet so that the magnets can adhere to the patient's skin around the wound.

In another embodiment, flexible sheet may be made of a shape retaining moldable material, such as a plastic material that retains its shape after being bent. Such materials include plastics or materials being made from polyethylene, polyvinyl chloride, polyurethane, or polypropylene.

In one embodiment the channel within the magnets is rectangular prism shaped, while in other embodiments the shape is triangular prism shaped, or cylindrical shaped.

In order to retain the magnets on the magnet receiving ends of the flexible sheet, the each magnet channel may have a plurality portions of different sizes so that the flexible sheet cannot be withdrawn in an upward direction, but can only be slidably disengaged from the magnets by sliding the flexible sheet in the elongate direction (i.e. horizontally) of the magnets, following the elongated direction of the channel within the magnets. In one embodiment the channel has an upper portion and a lower portion. The upper portion has a wider cross sectional width than the upper portion. The magnet receiving ends have a complimentary shape to the upper and lower channel portions and thereby cannot be withdrawn merely by pulling the flexible sheet up because the lower portion of the magnet receiving end is blocked from exiting the channel by the smaller width of the upper portion channel. In one embodiment both the upper and lower channel portions are rectangular shaped.

In another embodiment, there is only one channeled magnet, which is coupled to the first end the flexible sheet. On the second end of the flexible sheet, instead of a magnetic is channeled ferromagnetic structure. The ferromagnetic structure, while not a magnet itself, is attracted to the channeled magnet on the opposite side of the wound.

In an alternative embodiment embodiment, the magnet(s) are removably connected to the flexible sheet without the use of channels within the magnet, but may be removably connected inversely by the channels being on the ends of the flexible sheet. In another embodiment, the magnet(s) may be connected to the ends of the flexible sheet by snaps, hooks and loops (such as Velcro®), or connected via a material that has perforations between the magnet(s) and flexible sheet, where the magnets can then be removed by either pulling, or snapping, tearing, or the like, leaving only magnet(s) on the patient, and not the flexible sheet.

It is another object of the invention to provide a method of reapproximating wound edges, comprising the steps of adhering a first magnet of a wound closure assembly substantially near a first wound edge on a patient, adhering a second magnet of said wound closure assembly substantially near a second wound edge on a patient, said second wound edge substantially opposing said first wound edge, and uncoupling or removing the flexible sheet from the first magnet and second magnet.

The arrangement of the flexible sheet and channeled magnets in this wound closure assembly has several advantages over the prior art. First, there are no sutures involved. Second, there are few pieces of the assembly, making this assembly relatively inexpensive and easy to use. Third, the flexible sheet allows the practitioner hold the sheet, instead of the magnets direction, when placing the magnets on the patient. Fourth, the flexible sheet can be moldable and retain its molded position to help retain the magnets in a desired position. Fifth, and fifth, the sheet couples the magnets in a manner where the magnets do not easily disengage from the sheet when pulled inadvertently, but still provides easy an easy way for the practitioner to release the magnets from the flexible sheet.

### BRIEF DESCRIPTION OF THE DRAWINGS

These and other features and advantages of the present invention will become appreciated, as the same becomes better understood with reference to the specification, claims and drawings herein:

FIG. 1a is a side view of an assembled wound closure assembly having a flexible sheet and two magnets.

FIG. 1b is a side view of an assembled wound closure assembly curved for placement on a patient's wound.

FIG. 1c is an exploded view an assembled wound closure assembly having a flexible sheet and two magnets.

FIG. 2a is a side exemplary view of an assembled wound closure assembly placed around a patient's wound.

FIG. 2b is a side exemplary view of an assembled wound closure assembly placed around a patient's wound where the flexible sheet has been cut.

FIG. 3 is a side exemplary view of a wound closure assembly where the flexible sheet has been removed, leaving only the magnets around the patent's wound.

FIG. 4 is a top exemplary view of the wound closure assembly of FIG. 3.

### DETAILED DESCRIPTION OF THE INVENTION

The invention now will be described more fully hereinafter with reference to the accompanying drawings, in which embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art.

It will be understood that when an element is referred to as being “on” another element, it can be directly on the other element or intervening elements may be present there between. As used herein, the term “and/or” includes any and all combinations of one or more of the associated listed items.

It will be understood that, although the terms first, second, third etc. may be used herein to describe various elements, components, regions, layers and/or sections, these elements, components, regions, layers and/or sections should not be limited by these terms. These terms are only used to distinguish one element, component, region, layer or section from another element, component, region, layer or section.

The terminology used herein is for the purpose of describing particular embodiments only and is not intended to be limiting of the invention. As used herein, the singular forms “a,” “an,” and “the,” are intended to include the plural forms as well, unless the context clearly indicates otherwise. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Furthermore, relative terms, such as “lower” or “bottom” and “upper” or “top,” may be used herein to describe one element's relationship to another elements as illustrated in the Figures. It will be understood that relative terms are intended to encompass different orientations of the device in addition to the orientation depicted in the Figures.

Unless otherwise defined, all terms used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

Exemplary embodiments of the present invention are described herein with reference to idealized embodiments of the present invention. As such, variations from the shapes of the illustrations as a result, for example, of manufacturing techniques and/or tolerances, are to be expected. Thus, embodiments of the present invention should not be construed as limited to the particular shapes of regions illustrated herein but are to include deviations in shapes that result, for example, from manufacturing.

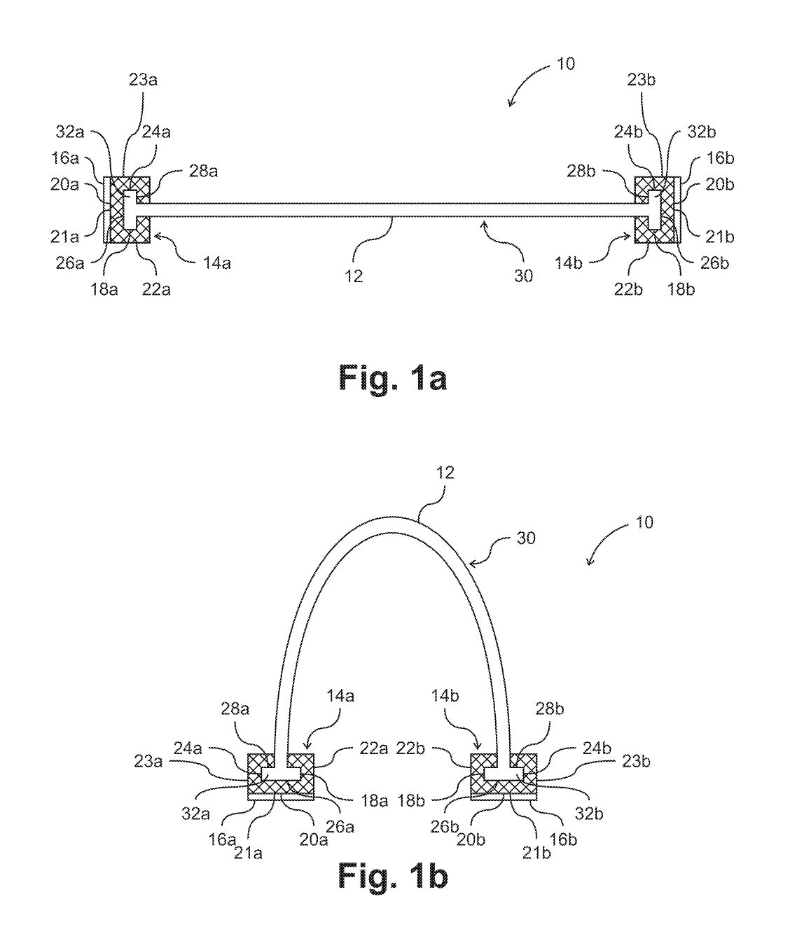

A wound closure assembly is provided for closing wounds without sutures. FIG. 1a and FIG. 1b depict one embodiment of a wound closure assembly 10. The assembly 10 has a flexible sheet 30 with two magnets 14a, 14b, one magnet on each of two ends 32a, 32b of the flexible sheet 30. The flexible sheet 30 has a central portion 12 that is flexible and can orient the magnets 14a, 14b in various directions, depending on how the practitioner bends the flexible sheet 30. The magnets 14a, 14b surround the ends 32a, 32b of the flexible sheet 30, such that the magnets 14a, 14b cannot be pulled off from the ends 32a, 32b in a perpendicular direction relative to the plane of contact surface 21a, 21b of each magnet 14a, 14b. Instead, the flexible sheet 30 can only disengage from the magnets 14a, 14b when the flexible sheet 30 slides in the lengthwise elongated direction channels 56a, 56b within each magnet 14a, 14b (as illustrated in FIG. 1C where the channels are shown in exploded view after the flexible sheet 30 has been removed). The magnets 14a, 14b cannot be removed merely by pulling the flexible sheet 30 away from the magnets because of at least one protrusion 28a, 28b on each magnet 14a, 14b directed toward the central axis of each magnet. This protrusion 28a, 28b prevents the flexible sheet ends 32a, 32b from being pulled out, and only allows the flexible sheet to be withdrawn in the lengthwise direction of each magnets 14a, 14b.

In the embodiment shown, each channel channels 56a, 56b has two portions, an upper channel portion 57a, 57b, and a lower channel portion 59a, 59b. The upper channel portion 57a, 57b, has a smaller cross sectional width than the cross sectional width of the lower channel portion 59a, 59b, thereby preventing the magnet coupling ends 32a, 32b from easily disengaging from the magnets 14a, 14b. Although the channel 56a, 56b shape depicted is rectangular prism shaped, its shape can vary. The shape of the magnet coupling ends 32a, 32b can be any shape that corresponds to the shape of the channels 56a, 56b, as long as each coupling ends 32a, 32b fits securely within each channel 56a, 56b. Examples of other channel shapes include channels that are cylindrically shaped, pyramidal shaped, hexagonal shaped, pentagonal shaped, or any shape that allows the upper channel portion to be less wide than the lower channel portion.

The purpose of the magnets 14a, 14b is to draw the edges of a wound close together. The magnets 14a, 14b each have 1) a contact surface 21a, 21b, having an adhesive bottom layer 20a, 20b, 2) a first side wall 23a, 23b, and 3) a second side wall 22a, 22b. Each side wall, 21a, 21b, 23a, 23b is approximately orthogonal to the contact surface 21a, 21b, of each magnet 14a, 14b. The contact surface 21a, 21b is pressed against the skin of the patient to adhere the magnets 14a, 14b around the wound of the patient. The adhesive may be made from a variety of compositions, including polyvinyl acetates, polyvinyl laminates, acrylates, polychloroprene, polyester resins, polyols, polyurethanes, or various other adhesives than can adhere a magnet safely to skin.

As illustrated in FIG. 1b, the second side wall 22a of the first magnet 14a is closest in proximity to the second side wall 22b of the second magnet 14b. When the magnets 14a, 14b are oriented on the flexible sheet 30 such that polarity of the first side wall 22a is opposite the polarity of the second side wall 22b, the two magnet side walls attract each other and draw in closer together, thereby reapproximating the wound edges since the magnets 14a, 14b are adhered to the skin. In another embodiment, only one structure coupled to the coupling end 32a is a magnet. The opposing structure on the second end of the flexible sheet 30 is ferromagnetic (but not a magnet itself). The opposing structure (which would substitute for second magnet 14b, but maintain the same physical structural form of the magnet 14b) is attracted to the first magnet 14a because the opposing structure would be made of a ferromagnetic element such as iron, nickel or cobalt which is attracted to the magnet 14a, and thereby also able to reapproximate wound edges.

In still another embodiment, the first magnet 14a and second magnet 14b are only magnetized (or have a magnetic structure) on one of the side walls 22a, 22b, but not the entire structure 14a, 14b, thereby still allowing the two structures 14a, 14b to attract each other and reapproximate a wound.

The magnets 14a, 14b adhere around the wound of a patient by having an adhesive layer 20a, 20b, on the contact surface 21a, 21b of each magnet 14a, 14b. In the embodiments shown in FIG. 1a and FIG. 1b, an adhesive cover 16a, 16b covers the adhesive layer 20a, 20b on the contact surface 21a, 21b. The adhesive cover 16a, 16b may be a peelable strip that remains on the magnets 14a, 14b during storage, and removed by the practitioner before placement on the patient.

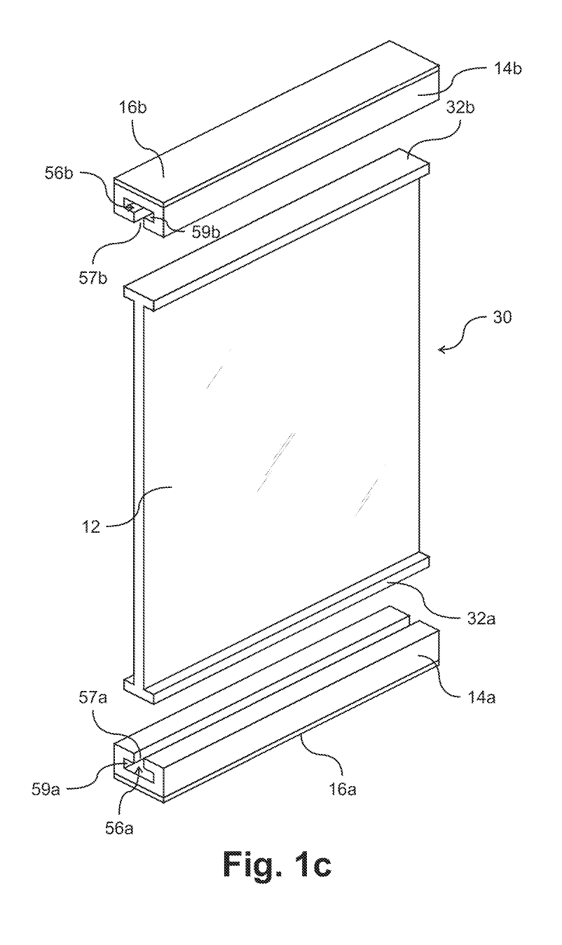

FIG. 2a depicts the wound closure assembly 50 of FIGS. 1a-c on a patient. The magnets 14a, 14b are placed on the skin 54 near a patient's wound 52 by placing the adhesive layer 20a, 20b of the magnets 14a, 14b on the skin 54. The magnets 14a, 14b may be placed on the periphery 62a, 62b of the wound, or in other embodiments, may be placed over the wound itself 52. In the embodiment illustrated in in FIG. 2b, the wound 52 may be so large that a single intact wound closure assembly would not large enough to place the magnets 14a, 14b around the wound 52. In this situation, the flexible strap 30 may be cut so that the magnets 14a, 14b can be placed farther apart, thereby producing a first flexible sheet 30a and a second flexible sheet 30b. The practitioner can also use several wound closure assemblies aligned next to each other to cover a large perimeter of a wound.

In any of the discussed embodiments, the practitioner uses the flexible sheet 30 to help guide and position the magnets around the wound 52. The flexible sheet 30 may be made of a resilient material or a shape-retaining moldable material such that when the sheet 30 is bent, the sheet 30 retains its shape, and the magnets 14a, 14b are held in position. Compositions of the flexible sheet 30 material may include a number of materials known in the prior art, such plastics, natural and synthetic rubbers, polyethylene, polyvinyl chloride, polyurethane, or polypropylene compositions. The magnets 14a, 14b may be made from a number of magnetic materials commonly used, including neodymium iron boron magnets, samarium cobalt magnets, alnico magnets, ceramic magnets, ferrite magnets, electromagnets and the like.

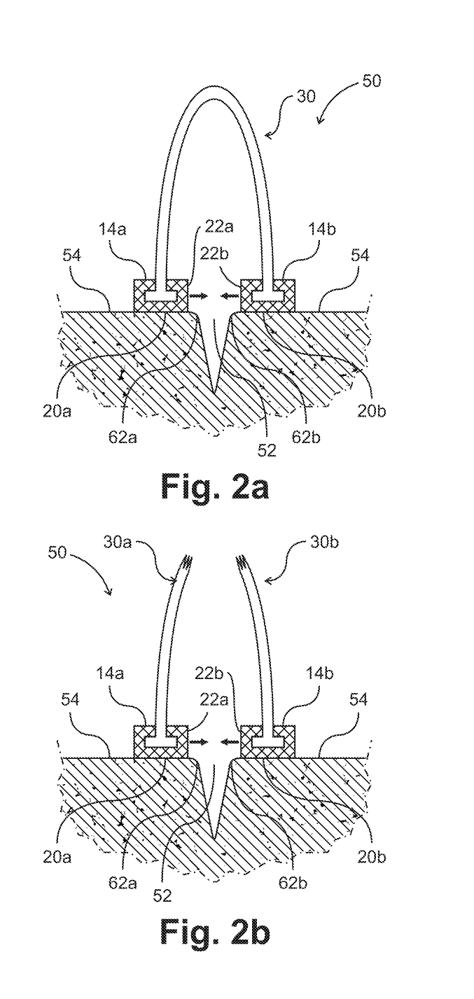

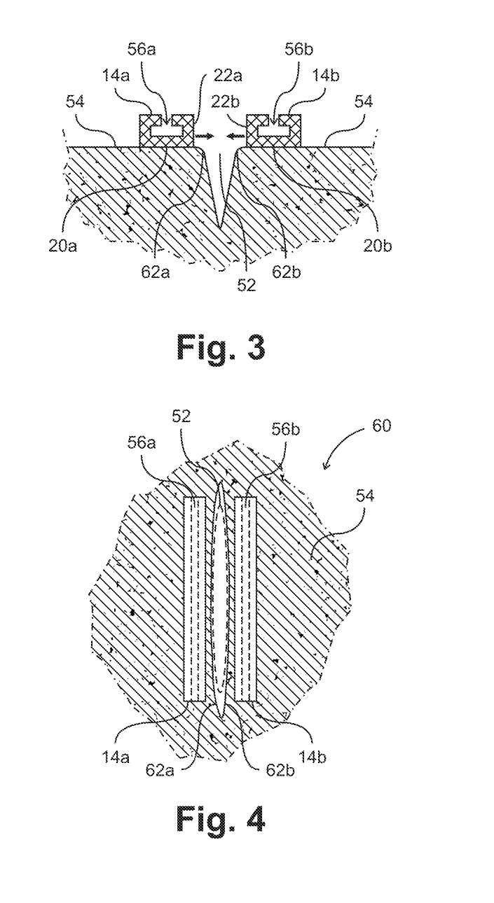

Once the magnets 14a, 14b, are placed around the wound 52, the practitioner can remove the flexible sheet 30 by sliding the sheet 30 in the lengthwise elongate direction of the channels 56a, 56b, leaving just the magnets 14a, 14b on the patient's skin 54 around the wound 52, as illustrated in side view on FIG. 3, and in top view in FIG. 4. As is readily apparent, the edges 62a, 62b of the wound 52 will be pulled together (i.e. reapproximated), thereby allowing a patient's wound 52 to heal quickly, and does not involve the use sutures, staples, or glue within the wound 52.

While the invention has been described in terms of exemplary embodiments, it is to be understood that the words which have been used are words of description and not of limitation. As is understood by persons of ordinary skill in the art, a variety of modifications can be made without departing from the scope of the invention defined by the following claims, which should be given their fullest, fair scope.

I claim:

1. A wound closure assembly comprising:
a) a flexible sheeting having
i) a first magnet connecting end
ii) a second magnet connecting end
iii) a central portion between said first end and said second end;
b) a first magnet having a first contact surface having a first adhesive layer; and,
c) a second magnet having a second contact surface having a second adhesive layer;
wherein said first magnet connecting end is removably connected to said first magnet, and said second magnet connecting end is removably connected to said magnet,
whereby said flexible sheet aids in placement and retention of said first and second magnets on opposite sides of a wound; and,
whereby said first and second magnet attract each when affixed to opposite sides of a wound on a patient, thereby drawing together opposite sides of the wound, resulting in wound end reapproximation.

2. The wound closure assembly of claim 1 further comprising a first and second removable adhesive cover, said first and second removable adhesive cover adjacent to said first and said second adhesive layer, respectively.

3. The wound closure assembly of claim 2 wherein said first and said second adhesive cover is a peelable strip.

4. The wound closure assembly of claim 1, further characterized by said flexible sheet being made from a shape-retaining moldable material.

5. The wound closure assembly of claim 1, further characterized by said flexible sheet being made from a plastic material.

6. The wound closure assembly of claim 1, further characterized by said flexible sheet being made from a polyethylene, polyvinyl chloride, polyurethane, or polypropylene material.

7. The wound closure assembly of claim 1 wherein said first magnet and said second magnet are substantially rectangular bar shaped.

8. The wound closure assembly of claim 1, wherein said first magnet and said second magnet are characterized by having a first channel and a second channel, respectively.

9. The wound closure assembly of claim 8, wherein said first channel and said second channel are rectangular prism shaped.

10. The wound closure assembly of claim 8, wherein said first channel and said second channel are triangular prism shaped.

11. The wound closure assembly of claim 8, where said first channel and said second channel are cylindrical shaped.

12. The wound closure assembly of claim 8, wherein said first channel has a first upper channel portion and a first lower channel portion, and wherein said second channel has a second upper channel portion and a second lower channel portion, said first and said second lower channel portion each having a cross-sectional width greater than a cross-sectional width of said first and second upper channel portion, respectively.

13. The wound closure assembly of claim 8, wherein said first magnet connecting end of said flexible sheet and said second magnet connecting end of said flexible sheet each has a greater cross-sectional width than said cross sectional width of said first and second upper channel portions, respectively, thereby allowing slidable disengagement from said first and said second magnets.

14. A wound closure assembly comprising:
a) a flexible sheet having
i) a first end for connecting a magnet,
ii) a second end for connecting a ferromagnetic structure, and
iii) a central portion between said first end and said second end,
b) a magnet having
i) a first channel, and
ii) a first contact surface having a first adhesive layer, and,
c) a ferromagnetic structure having
i) a second channel,
ii) a second contact surface having a second adhesive layer, and,
wherein said first and second ends of said flexible sheet are correspondingly shaped to said first and second channels, respectively, and
wherein said first end is connected to said magnet, and said second end is connected to said ferromagnetic structure, and
whereby said flexible sheet aids in placement and retention of said first and second magnets on opposite sides of a wound; and,
whereby said magnet attracts said ferromagnetic structure when affixed to opposite sides of a wound on a patient, thereby drawing together opposite sides of the wound, resulting in wound edge reapproximation.

15. A method of reapproximating wound edges, comprising the steps of:
a) adhering a first magnet of a wound closure assembly substantially near a first wound edge on a patient, said wound closure assembly comprising,
i) a flexible sheet having a first magnet connecting end, a second magnet connecting end, and a central portion between said first end and said second end,
ii) said first magnet having a first adhesive layer, and said second magnet having a second adhesive layer,
wherein said first magnet connecting end is connected to said first magnet, and said second magnet connecting end is connected to said second magnet,
a) adhering said second magnet of said wound closure assembly substantially near a second wound edge on a patient, said second wound edge substantially opposing said first wound edge; and,
b) removing said flexible sheet from said first magnet and said second magnet,
whereby said first magnet and said second magnet attract each, thereby reapproximating wound edges.

16. The method of claim 15 further comprising the step of curving said middle portion of said flexible sheet to bring said first and said second magnets in proximity to each other.

17. The method of claim 15, wherein said first and second magnets are further characterized by having a first channel and a second channel, respectively, said first magnet connecting end is connected to said first channel, said second magnet connecting end is connected to said second channel, and, wherein removing said flexible sheet is accomplished by sliding said first and second magnet from said first and said second channels.

18. The method of claim 15, wherein adhering said second magnet is further characterized by adhering said second magnet substantially parallel to said first magnet.

19. The method of claim 15, wherein said wound closure assembly further comprises a removable adhesive cover, and further comprising the step of removing said removable adhesive cover.
